(19) United States
(12) Patent Application Publication
(10) Pub. No.: US 2003/0073089 A1
(43) Pub. Date: Apr. 17, 2003

(54) Companion cartridge for disposable diagnostic sensing platforms

(76) Inventors: Ganapati R. Mauze, Sunnyvale, CA (US); Michael Greenstein, Los Altos, CA (US); Catherine K. Templin, Portola Valley, CA (US); Paul Lum, Los Altos, CA (US); Leslie Leonard, Portola Valley, CA (US); Dirk Boecker, Palo Alto, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 09/982,307

(22) Filed: Oct. 16, 2001

(51) Int. Cl. C12Q 1/68; C12P 19/34; C12M 1/34

(52) U.S. Cl. 435/6; 435/287.2; 435/91.2

(57) ABSTRACT

A method and apparatus comprising a companion cartridge containing a reagent storage system, a calibration fluid system, an assay element storage system, a carrier fluid system, and a waste retrieval system connected to a sensing cartridge to conduct chemical analysis. The companion cartridge can be adapted for assays related to blood chemistry, hematology, immuno-diagnostics, and DNA testing.

### DESCRIPTION OF THE INVENTION

1. Field of the Invention

The present invention relates to an apparatus and method for chemical and biological analysis. More particularly, to analyzing body fluids, such as blood samples. The analysis can be carried out on an sensing cartridge. A companion cartridge can be adapted to carry out unit operations such as reagent storage, calibration, assay preparation, carrier fluid transport, and waste retrieval. The companion cartridge, thereby, facilitates detection by a separate sensing cartridge.

2. Background of the Invention

Clinical chemistry involves the qualitative and quantitative analyses of body fluids, such as blood, urine, spinal fluid, and other materials. Clinical chemistry encompasses multiple specialty testing areas including coagulation, hematology, immunochemistry, as well as chemistry. The test results derived from such analyses can be used by physicians and other healthcare professionals to diagnose, monitor, and treat diseases. The analysis protocols, instrumentation, and other equipment utilized in clinical laboratory testing can provide accurate and repeatable test results. In addition, the procedures and instrumentation can be simple, efficient, and versatile that patients can use these to self-monitor outside of the clinical setting.

The analysis and quantification of blood components is an important diagnostic tool for better understanding the physical condition of a patient. Since current devices and methods do not provide for complete blood analysis on the miniature scale, blood samples still need to be sent to laboratories for complete analysis. Otherwise, a patient may self-administer a test which may give results for one blood analyte. A well known example of such analysis is self-monitoring of glucose levels by a diabetic individual performed at home.

Many products for self-monitoring of blood glucose levels are available commercially. Upon doctors' recommendations and using such products, patients typically measure blood glucose level several (3-5) times a day as a way to monitor their success in controlling blood sugar levels. For many diabetics, the failure to test blood glucose regularly may result in damage to tissues and organs, such as kidney failure, blindness, hypertension, and other serious complications. Nevertheless, many diabetics do not measure their blood glucose regularly. Similar risks exist with other health conditions. These risks require monitoring particular blood analytes as indicative of deteriorating health, and alerting the patient to seek treatment or modify lifestyle. Patients do not regularly monitor these additional indicators because the existing monitoring products may be complicated, inconvenient, and painful, requiring a pinprick every time one measurement is made. Otherwise, the patient has to visit a physician or phlebotomist to draw blood for complete analysis. Furthermore, self-administered test products require some skill, dexterity, and discipline to obtain useful measurements. Such instruments require a calibration step followed by transport of the blood sample to the fill port for testing. This involves insertion of a blank or a waiting period for an internal calibration by the analytical instrument.

Self-administered lancing of the skin to obtain blood typically yields a small droplet of blood with a volume of 2-20 micro-liters. Accordingly, there has been a trend in clinical chemistry to develop analytical systems which are capable of conducting numerous different chemical analyses on these small samples, so that the maximum number of medical tests can be made using the minimum amount of sample. Attempts to force the bulk fluid-handling and sensing on a common platform to conduct a battery of tests results in high cost of manufacture, decreased yield from the fabrication processes, and compromises in functionality, performance, and shelf-life.

The present invention addresses these challenges since it describes an apparatus and a method of fluid management for analytical testing, which offers unit operations (such as reagent storage, calibration, assay element preparation, carrier fluid transport, and waste retrieval) on a companion cartridge, separate from the operations of a physically distinct sensing cartridge. The sensing cartridge handles the sample and a minimum volume of reagents and calibration fluid to conduct a majority of the desired chemical analysis. The term “companion cartridge” refers to a cartridge that comprises additional volume for unit operations to conduct the chemical analysis on the sensing cartridge. The additional volume comprises volume for fluids including reagent, calibration, and carrier fluids, and volume for assay element preparation and waste retrieval. The term “sensing cartridge” refers to a cartridge which can conduct chemical analysis on several parameters of a body fluid sample, and comprises sufficient reagent and calibration fluids for a majority of the desired types of chemical analysis. The sensing cartridge can couple with the companion cartridge to increase the number of parameters which can be tested by increasing the types of chemical analysis possible on the sensing cartridge by adding volume for fluids and assay preparation. This distinction between the cartridges allows for better preservation of the chemicals required for some types of chemical analysis, which may not be run by every user or during each use of the instrument. This is important because 50% of the desired diagnostic tests run on blood do not require large volumes of blood, reagents, or calibration fluid. Such tests can be run on the sensing cartridge, without requiring significant storage and preparation volume. Another 20% of the desired blood diagnostics are for coagulation, and a further 10% for hematology. These tests that are prescribed only a minority of times require proportionally larger volumes of fluids which can be stored on the companion cartridge. Since these fluids are needed only 10-20% of the time, the companion cartridge can be coupled to the sensing cartridge only on the occasions when these types of tests are desired.

The invention comprises configuring several systems for storage space, structures and mechanisms in the companion cartridge to conduct the unit operations including reagent storage, calibration, assay preparation, carrier fluid transport, and waste retrieval. A reagent storage system comprises additional reagents or excess reagents already contained on the sensing cartridge. A calibration fluid system comprises additional calibration fluid or excess calibration fluid already contained on the sensing cartridge. An assay element preparation system conducts additional preparation of the assay elements (including reagents stored on the companion cartridge and body fluid sample drawn from the sensing cartridge) to conduct operations such as thermo cycling, incubation, and isolation involved in lysing, DNA isolation, and PCR processes. A carrier fluid system comprises a non-reactive carrier fluid to manipulate the assay elements for operations such as flow cytometry involved in hematology processes. A waste retrieval system retrieves the excess volume for operations requiring a flow of fluid (such as flow cytometry) involved in hematology processes.

### SUMMARY OF THE INVENTION

In accordance with the invention, an apparatus and method for chemical and biological analysis offers unit operations (such as reagent storage, calibration, assay element preparation, carrier fluid, and waste retrieval) on a companion cartridge in communication with a sensing cartridge.

The invention provides a method and a companion cartridge containing an assay element storage system, a calibration fluid system, an assay element storage system, a carrier fluid system, and a waste retrieval system connected to the sensing cartridge. The term “assay elements” refers to body fluid samples and reagents for the assay. The calibration fluid system provides additional calibration fluid for tests, where the volume on the sensing cartridge would be prohibitive for calibrating the detectors on the instrument used in conjunction with the sensing cartridge. Some reagents can be used as calibration fluid. The assay element preparation system performs operations such as thermo cycling, incubation, and isolation involved in lysing, DNA isolation, and PCR processes. The carrier fluid system assists in the manipulation of the assay elements using a non-reactive carrier fluid. The waste retrieval system is capable of retrieving the excess volume for operations requiring a flow of fluid such as flow cytometry involved in hematology processes.

Diagnostic tests based on disposable companion cartridges can include blood chemistry, hematology, immuno-diagnostics, and DNA testing. Typically, in these types of tests, the volume of reagents used and the amount of waste generated is often considerably larger than the volume of body fluid actually used for testing. The sensing cartridge is of small size, i.e. 20-50 micro-liters liquid volume capacity, and can handle the volume of a body fluid sample and some reagents necessary for a majority of the desired chemical analysis, which do not require proportionally large volumes of reagent and calibration fluid. Whereas, the companion cartridge can house the larger volume (i.e. 50-500 micro-liters liquid volume capacity) for operations such as flow cytometry, which require larger volumes of carrier fluid. The term “miniature” refers to chemical analysis of volumes 0-1000 micro-liters.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates an embodiment of the sensing cartridge showing the sensing cartridge with assay element handling and sensors.

FIG. 2 illustrates an embodiment of the companion cartridge showing a calibration fluid, carrier fluid, or reagent storage system overlapping the sensing cartridge.

FIG. 3 illustrates an embodiment of the companion cartridge showing the companion cartridge with an assay element preparation system.

FIG. 4 illustrates an embodiment of the companion cartridge containing a waste retrieval system.

In FIGS. 2 and 5, the overlapping companion cartridge systems are shown in solid lines and the sensing cartridge is shown in broken lines.

FIGS. 5a-5d illustrate an embodiment of the companion cartridge with the sensing cartridge from the top view, side view, end view, and alternate end view, respectively.

### DETAILED DESCRIPTION OF THE INVENTION

This invention provides a companion cartridge that contains the space, structures and mechanisms to perform unit operations before, during, and after the assay necessary to position the assay elements in the active area of the sensor cartridge. The companion cartridge contains the unit operations, but also can interface with the instrument mechanically, fluidically, or electronically. The term “system” refers to the unit operations involved in fluid management on the cartridge including storage of reagent, calibration, and carrier fluids, and preparation of assay elements and waste retrieval.

Unit operations on the companion cartridge store, prepare, and retrieve assay elements, carrier fluids and calibration fluids. The companion cartridge can incorporate a power source, and control composition, volume, temperature, and pressure of assay elements and calibration fluids. The signals for such control can be relayed to the companion cartridge through electronic, optical, pressure, or radiofrequency (RF) communication means, or pre-programmed into a non-volatile memory incorporated in the companion cartridge.

Unit operations typically require transport of the fluid of interest. This invention contemplates active and passive transport, including active and passive microfluidics. An example of active transport is paddle wheel pumping as described in a copending application (Attorney Docket Number 10004024, Inventor: Paul Lum, entitled “A MICRO PADDLE WHEEL PUMP FOR THE PRECISE PUMPING, MIXING, DISPENSING, AND VALVING OF BLOOD AND REAGENTS”) assigned to the same assignee as the present application. Said copending application is incorporated by reference in its entirety herein.

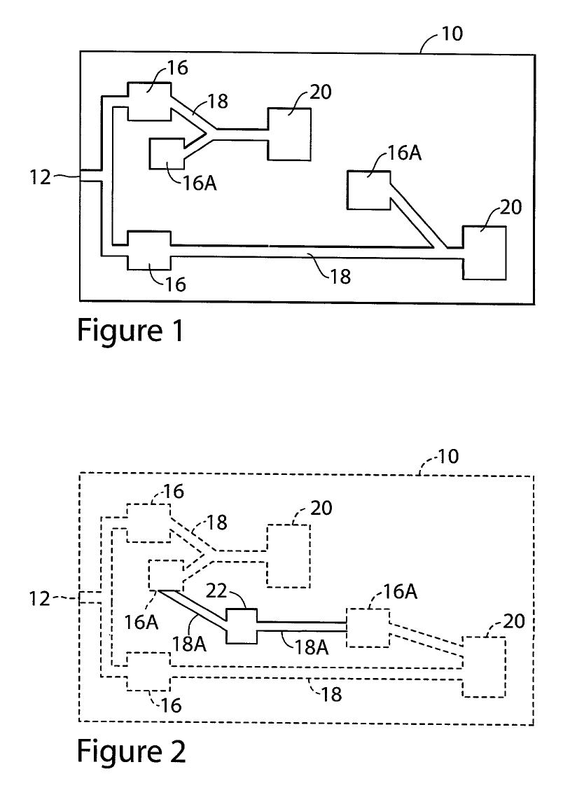

Reference will now be made in detail to the exemplary embodiments of the invention. FIG. 1 illustrates an embodiment of the sensing cartridge. The term “assay element” refers to body fluid samples (such as blood), reagent chemicals, and analytes, which can support a variety of analytical methods, including electrochemical, chemiluminescence, optical, electrical, mechanical and other methods. Blood chemistry tests such as blood gasses (including pO₂, pCO₂), blood pH, hematology, hematocrit and coagulation and hemoglobin factors, as well as immuno-diagnostics, and DNA testing, ions (Na⁺, Ca⁺⁺, K⁺), and small molecules such as glucose and lactate can be performed on the sensing cartridge. The sensing cartridge (10) contains a system of body fluid accumulation reservoirs (16), reagent or calibration fluids reservoirs (16A), tubes (18), and assay active areas (20). The body fluid sample is introduced into the system through to entry port (12). The body fluid accumulation reservoirs (16), which contain these samples, are connected to entry port (12) through tubes (18). Reagent or calibration fluids reservoirs (16A) contain assay elements stored during the manufacture of the sensing cartridge (10) and are connected to assay active areas (20) through tubes (18). The term “connect” or “connecting” refers to using plumbing for attachment of components of a system or different systems. The term “system” refers to at least one space, structure, or mechanism for fluid management on the companion cartridge. The assay elements in reservoirs (16) and (16A) are transported through tubes (18) to the assay active areas (20) where the assay elements can be analyzed by the detectors on the instrument.

FIG. 2 illustrates an embodiment of the companion cartridge, comprising a calibration system, a reagent storage system, or carrier fluid system. The companion cartridge (not shown) comprises pre-packaged additional or excess reagent, calibration, or carrier fluids in storage reservoirs (22). The fluid can be transported to the sensor cartridge (10) through fluid connecting tubes (18A) and collected in reservoirs (16A). In a calibration system, calibration fluid transported through fluid connecting tubes (18A) can calibrate the detectors associated with assay active areas (20). Calibration fluid reaches and fills the assay active area (20) to calibrate the detector on the instrument prior to introduction of the assay elements into assay active area (20). Alternatively, the companion cartridge can be in direct fluid contact with the active areas (20) without passing through reservoir (16A). The calibration fluid can be displaced into the waste retrieval system after calibration is complete to avoid interference with the assay element measurement. Alternatively, calibration may occur by mixing two or more fluids stored in the companion cartridge calibration fluid system. Such a configuration allows single point or multipoint calibration or referencing. A reagent storage system operates similarly to the calibration system.

A carrier fluid can be used to aliquot from a large volume of assay element. The carrier fluid can be used to fill up the dead space, so that a small aliquot can be moved around within the microfluidic systems without need for the whole system to be filled by the assay element. This allows testing of small assay element volumes in relatively larger volume systems. The carrier fluid can be designed for calibration, referencing, or for regenerating the active area between tests. In microfluidic systems, the flow of fluids is usually laminar where the main cause of mixing is diffusion. Thus, the amount of mixing between the carrier fluid and the assay element would be low, and assay element fidelity is essentially preserved. An example of chemical analysis using carrier fluid is flow cytometry blood count in hematology.

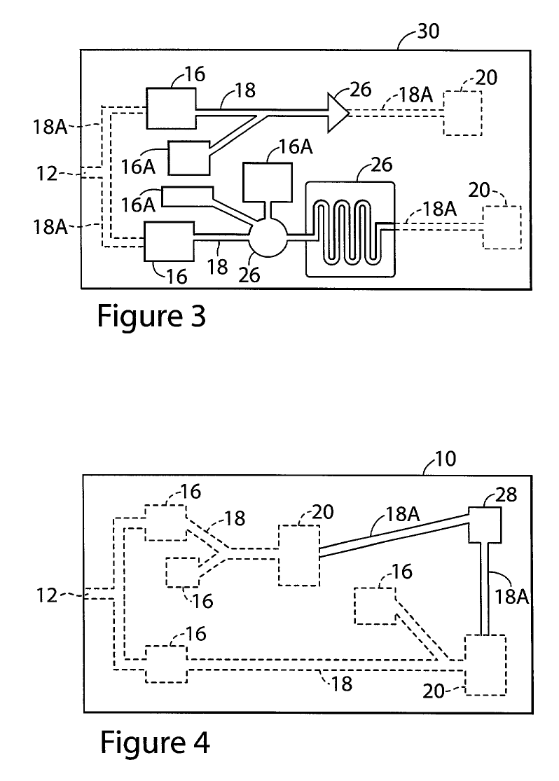

FIG. 3 illustrates an embodiment of the assay element preparation system. The companion cartridge (30) contains operators (26) to conduct a process for assay element preparation. Some operators may require thermal regulation of the region around these operators (26). Examples of such localized thermal regulation are described in a copending application (Attorney Docket Number 10004416, Inventors: Frederick Stawitcke, et al., entitled “METHOD OF THERMAL REGULATION OF FLUIDIC SAMPLES WITH A DIAGNOSTIC CARTRIDGE”) assigned to the same assignee as the present application. Said copending application is incorporated by reference in its entirety herein. The body fluid sample can be transported from entry port (12) to body fluid accumulation reservoirs (16) through fluid connecting tubes (18A). The body fluid can be then be transported to operators (26) through tubes (18). Reagent or carrier fluid reservoirs (16A) can be connected to operators (26) through tubes (18). The operators (26) prepare the assay elements for measurements of blood chemistries, hematology, immuno-diagnostics, and DNA testing and then can be transported to assay active areas (20) through fluid connecting tubes (18A). The term “process” refers to any assay element preparation necessary to conduct measurements in blood chemistry (arterial blood gases, electrolytes, metabolites, coagulation), hematology, immuno-diagnostics, or DNA testing. Each process includes operations, such as cell lyses, isolation of specific components such as DNA, or amplification of a sample through PCR. The term “operation” refers to a specific task necessary to conduct a process such as thermo cycling, incubation, or isolation. For example the operator (26) mixes the primers with PCR reagents in proper order and amounts. The operations for each process are known in the art, as is the means of conducting such operations on the miniature scale, for example as is described in “Handling Fluids in Microsensors,” Science & Technology Review, Lawrence Livermore National Laboratory, November 1999. This reference is incorporated by reference in its entirety herein.

FIG. 4 illustrates an embodiment of the companion cartridge comprising a waste retrieval system. The companion cartridge (not shown) comprises a waste retrieval system comprising the waste reservoir (28). Some assays require the use of relatively large volumes of fluid, as compared to the volume of body fluid sample. The waste retrieval system can retrieve through fluid connecting tubes (18A) and transport to waste reservoir (28) the excess fluid that flows through assay active areas (20). An example of such an assay is blood count via flow cytometry. The excess carrier fluid required to align the red blood cells flows through the assay active area (20) into the waste reservoir (28). Alternatively, the waste retrieval system can be connected to the reagent, calibration, or carrier fluid system, such that the waste retrieval system uses the reservoirs (22) as waste reservoirs. Such a configuration has the added benefit of displacing the fluid stored in reservoir (22), thereby facilitating the passive transport of the fluid. The waste retrieval system may accept fluids from the calibration system, or the carrier fluid system, as well as the assay elements.

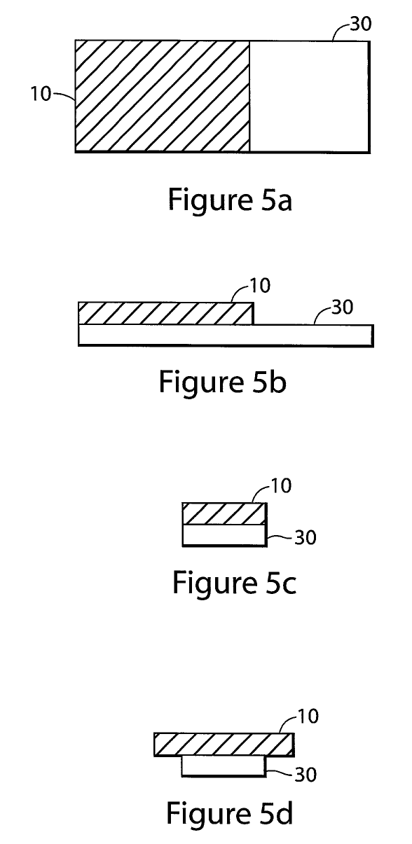

FIG. 5a through 5d illustrate an embodiment of the companion cartridge with the sensor cartridge from the top view, side view, end view, and alternate end view, respectively. The companion cartridge (10) and sensor cartridge (30) can overlap and protrude in a variety of ways to allow the instrument to detect results from the sensors on the sensor cartridge (30). In one embodiment, the companion cartridge (10) and sensor cartridge (30) can be fit together prior to insertion into the instrument. In another embodiment, the two cartridges can be placed separately into the instrument. The term “instrument” or “analytical instrument” refers to a portable hand-held device with at least one slot to receive sensor cartridges and/or companion cartridges. This device contains detectors aligned with assay active areas on the sensing cartridge for quantifying the results of the assay.

The companion cartridge can be designed to fit one or more instruments and interact with one or more types of sensing cartridges. The design features permit changing and switching the sensing cartridge, without having to replace the companion cartridge. This is because the companion cartridge is removable, and does not need to be attached to the sensor cartridge for operation with the instrument. Such efficiency can reduce the cost of the sensing cartridge by the cost of the materials contained on the companion cartridge. This is because the companion cartridge does not need to be discarded along with the sensing cartridge when an assay that does not necessitate the companion cartridge is run. In other embodiments, the companion cartridge and/or the sensing cartridge can be reusable, allowing for several companion cartridges to be used with one sensing cartridge, or several sensing cartridges to be used with one companion cartridge. The earlier embodiment allows a blank companion cartridge to be used exclusively to calibrate the sensing cartridge. The latter embodiment allows multiple sensors to analyze one sample of body fluid. Additionally, such modularity allows the independent manufacture of the companion cartridge and sensor cartridge.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A companion cartridge comprising at least one system chosen from reagent storage system, a calibration fluid system, an assay element preparation system, a carrier fluid system, and a waste retrieval system,
said system connected to a sensing cartridge to conduct chemical analysis of at least one assay element.

2. A companion cartridge according to claim 1, wherein:
said companion cartridge is adapted to at least one interface with said sensing cartridge chosen from fluid, mechanical, and electrical.

3. A companion cartridge according to claim 1, wherein:
said calibration fluid system connects to said waste retrieval system.

4. A companion cartridge according to claim 1, wherein:
said assay element preparation system comprises at least one process chosen from blood chemistry, hematology, immuno-diagnostics, and DNA testing.

5. A companion cartridge according to claim 1, wherein:
said assay element preparation system comprises at least one operation chosen from thermo cycling, incubation, and isolation.

6. A companion cartridge according to claim 1, wherein:
said carrier fluid system adapted to increase at least a portion of the volume of at least one assay element.

7. A companion cartridge according to claim 6, wherein:
said carrier fluid system connects to said waste retrieval system.

8. A companion cartridge according to claim 6, wherein:
said carrier fluid remains essentially unmixed with said assay element.

9. A method of miniature chemical analysis comprising:
at least one unit operation chosen from:
storing at least one reagent on a companion cartridge;
calibrating at least one sensor on a sensing cartridge with calibration fluid stored on said companion cartridge;
preparing said at least one assay element for said assay;
transporting said at least one of said assay elements with a carrier fluid; and
retrieving waste from said assay
wherein said sensing cartridge is in fluid contact with said companion cartridge.

10. A method of miniature chemical analysis according to claim 9, wherein:
said preparing comprises at least one process chosen from blood chemistry, hematology, immuno-diagnostics, and DNA testing.

11. A method of miniature chemical analysis according to claim 10, wherein: said process comprises lysis of said assay element.

12. A method of miniature chemical analysis according to claim 10, wherein: said process comprises isolating DNA from said assay element.

13. A method of miniature chemical analysis according to claim 10, wherein:
said process comprises amplifying said assay element with PCR.

14. An analytical instrument comprising:
a companion cartridge comprising at least one unit operation chosen from a reagent storage system, a calibration fluid system, an assay element preparation system, a carrier fluid system, and a waste retrieval system; and
a sensor cartridge, said sensor cartridge in fluid contact with said companion cartridge, wherein said sensor cartridge provides chemical analysis of at least one assay element.

15. An analytical instrument according to claim 14, wherein:
said chemical analysis comprises at least one clinical process chosen from blood chemistry, hematology, immuno-diagnostics, and DNA testing.

16. An analytical instrument according to claim 14, wherein:
said chemical analysis comprises lysis of at least one assay element.

17. An analytical instrument according to claim 14, wherein:
said chemical analysis comprises isolating DNA from at least one assay element.

18. An analytical instrument according to claim 14, wherein:
said chemical analysis comprises amplifying at least one assay element with PCR.

19. A miniature analytical instrument comprising:
a sensor cartridge for chemical analysis of at least one assay element adapted to fluid contact with a removable companion cartridge.

20. A miniature analytical instrument according to claim 19, further comprising:
said companion cartridge in fluid contact with said sensor cartridge, said companion cartridge comprising at least one system chosen from reagent storage system, a calibration fluid system, an assay element preparation system, a carrier fluid system, and a waste retrieval system.
